(19) United States
(12) Patent Application Publication
(10) Pub. No.: US 2023/0214904 A1
(43) Pub. Date: Jul. 6, 2023

(54) METHOD AND APPARATUS FOR DISTRIBUTING COSMETICS FOR USERS BASED ON MANUFACTURERS AND DISTRIBUTORS WHO USE COMPREHENSIVE COSMETICS PROVIDING SERVICES

(71) Applicant: LULULAB INC., Seoul (KR)

(72) Inventor: Yongjoon CHOE, Seoul (KR)

(21) Appl. No.: 17/733,013

(22) Filed: Apr. 29, 2022

(30) Foreign Application Priority Data
Jan. 5, 2022 (KR) 10-2022-0001686

(51) Int. Cl. G06Q 30/06; A61K 8/00; G06V 40/16

(52) U.S. Cl. CPC G06Q 30/0631; A61K 8/00; G06V 40/169; A61K 2800/10

(57) ABSTRACT

Provided is a comprehensive cosmetic provision platform server for providing a service that recommends a cosmetic to a user, the comprehensive cosmetic provision platform server including: a manufacturer management unit configured to conclude a contract with a manufacturer server and to obtain information about a manufacturer and information about the cosmetic provided by the manufacturer from the manufacturer server; a distributor management unit configured to conclude a contract with a distributor server and to obtain distributor information from the distributor server; a data management unit configured to be interlocked with the comprehensive cosmetic provision platform server and to obtain a face image of the user from a kiosk installed in a distribution store of the distributor server; a skin characteristic determination unit configured to determine skin characteristics of the user based on the obtained face image; a cosmetic determination unit configured to determine a cosmetic including an ingredient and effect necessary for the user based on the determined skin characteristics; a kiosk control unit configured to display information about the determined cosmetic through the kiosk; a payment processing unit configured to obtain a payment request signal for the cosmetic from the kiosk and to perform payment for the cosmetic according to the obtained payment request signal; and a settlement processing unit configured to settle a sales amount for the cosmetic and to charge a fee to the manufacturer server and the distributor server based on the sales amount.

### FIELD OF INVENTION

The present invention relates to a method of distributing cosmetics for users based on manufacturers and distributors using comprehensive cosmetic provision services; and an apparatus therefor.

### BACKGROUND OF INVENTION

As interest in beauty increases, interest in skin beauty of the face is recently increasing. In addition, the consumption propensity of today's buyers is markedly different from that of the past. Unlike conventional buyers who directly view and purchase products offline, today's buyers prefer to purchase various products through communication networks using devices such as computers or smartphones. In this case, purchasers of cosmetics can obtain cosmetic information by referring to a cosmetics shopping mall, blog, and product reviews of products.

In particular, skin condition measurement devices that captures the user's facial skin and analyzes various skin characteristics (e.g., wrinkles, pores, acne, etc.) on the user's face are being developed. Through this, the user's skin condition can be measured quickly and accurately, and cosmetics for the user may be recommended according to the user's skin condition.

However, it is difficult to grow the cosmetic market by simply and uniformly recommending pre-registered products according to skin conditions by means of a skin condition measurement device. To further increase the demand of potential or prospective customers who want to purchase cosmetics, it is necessary to establish a cooperative system with manufacturers and distributors.

### SUMMARY OF INVENTION

### Technical Problem to be Solved

Therefore, the present invention has been made in view of the above problems, and it is one object of the present invention to provide a method of providing a service for diagnosing skin condition and a product recommendation service to customers using various types of stores through a skin diagnosis kiosk.

It is another object of the present invention to provide a method for promoting sales of cosmetics in conjunction with multiple manufacturers and distributors.

It is yet another object of the present invention to provide a commission system for multiple manufacturers and distributors.

### Technical Solution

In accordance with an aspect of the present invention, the above and other objects can be accomplished by the provision of a comprehensive cosmetic provision platform server for providing a service that recommends a cosmetic to a user, the comprehensive cosmetic provision platform server including: a manufacturer management unit configured to conclude a contract with a manufacturer server and to obtain information about a manufacturer and information about the cosmetic provided by the manufacturer from the manufacturer server; a distributor management unit configured to conclude a contract with a distributor server and to obtain distributor information from the distributor server; a data management unit configured to be interlocked with the comprehensive cosmetic provision platform server and to obtain a face image of the user from a kiosk installed in a distribution store of the distributor server; a skin characteristic determination unit configured to determine skin characteristics of the user based on the obtained face image; a cosmetic determination unit configured to determine a cosmetic including an ingredient and effect necessary for the user based on the determined skin characteristics; a kiosk control unit configured to display information about the determined cosmetic through the kiosk; a payment processing unit configured to obtain a payment request signal for the cosmetic from the kiosk and to perform payment for the cosmetic according to the obtained payment request signal; and a settlement processing unit configured to settle a sales amount for the cosmetic and to charge a fee to the manufacturer server and the distributor server based on the sales amount.

In accordance with various embodiments, the settlement processing unit may settle a first sales amount for the cosmetic paid according to the payment request signal; and a second sales amount for the cosmetic paid through a payment terminal of the distributor, and the second sales amount may be a sales amount in which payment for the cosmetic is performed the payment terminal within a predetermined time after information about the cosmetic is displayed through the kiosk.

In accordance with various embodiments, the comprehensive cosmetic provision platform server may further include a smooth distribution area determination unit for determining a smooth distribution area, which is a smoothly distributable area, within a range that can be distributed by the distributor, the cosmetic determination unit may determine the cosmetic based on cosmetic use information of other users who are distinguished from the user and who reside in the smooth distribution area of the distributor, the smooth distribution area determination unit may determines a distribution access degree of the distribution store based on a time taken from the time the cosmetic is paid to collection processing, trunk loading, trunk unloading, delivery, and delivery completion of the cosmetic, and may determine an area, in which the determined distribution access degree is a threshold access degree or more, as the smooth distribution area.

In accordance with various embodiments, the comprehensive cosmetic provision platform server may further include a manufacturer fee determination unit, wherein the manufacturer fee determination unit determines the manufacturer fee for the manufacturer server based on a monthly average sales ranking, selling price, and margin rate of the cosmetic payment-requested by the user.

In accordance with various embodiments, the comprehensive cosmetic provision platform server may further include a distributor fee determination unit, wherein the distributor fee determination unit determines a distributor fee for the distributor server based on the settled first and second sales amounts, a product relevance degree between the cosmetic and other products that are being sold in the distribution store and distinguished form the cosmetic, and utilization of the kiosk.

In accordance with various embodiments, the distributor fee determination unit may determine may determine utilization of the kiosk based on a gross sales growth/decrease rate of the distribution store calculated based on the time that the kiosk was installed in the distribution store, the number of face images obtained through the kiosk during a present period, an adoption rate of cosmetics recommended by the kiosk, an installation position of the kiosk in the distribution store, and the like, and may determine the distributor fee to be lower than a preset distributor fee as the kiosk utilization is high.

In accordance with various embodiments, the distributor fee determination unit may determine the product relevance degree between the cosmetic and other products based on the types of the other products that are distinguished from the cosmetic and are being sold at the distribution store, and may determine may determine the distributor fee to be lower than a preset distributor fee when the determined product relevance degree is a threshold relevance degree or more.

In accordance with various embodiments, the distributor fee determination unit may determine a product relevance degree between each of the other products between a cosmetic based on whether the other products are food and beverage products, fluid components-containing products, skin care-related products, medical products, beauty-related products, or health-related products. The distributor fee determination unit may calculate a first score according to whether the other products are food and beverage products, a second score according to whether the other products are fluid components-containing products, a third score according to whether the other products are skin care-related products, a fourth score according to whether the other products are medical products, a fifth score according to whether the other products are beauty-related products, and a sixth score according to whether the other products are health-related products, and may determine a product relevance degree based on the first to sixth scores.

In accordance with various embodiments, the smooth distribution area determination unit may determine the distribution access degree through Equation below:

$${S}_{a} = \{ d + \frac{{\sum }_{k = 1}^{j} ( {a}_{k} \times x + {c}_{k} \times y + {l}_{k} \times z )}{j} \} \times N$$

where Sₐ denotes the distribution access degree, Sd denotes a time taken from collection processing of cosmetics to loading on a trunk line, a denotes the length of an arterial road among distribution routes of the k-th cosmetics, c denotes the length of a collection road among distribution routes of the k-th cosmetics, l denotes the length of a local road among distribution routes of the k-th cosmetics, and j denotes the number of deliveries of cosmetics. N is an access factor and may vary depending on the ordered time and day of the week for cosmetics.

### Effect of Invention

In accordance with various embodiments of the present specification, user's skin characteristics can be diagnosed using a user's face image.

In addition, according to various embodiments, sales of cosmetics suitable for users can be promoted by installing a skin analysis kiosk in many distribution stores.

Further, various effects directly or indirectly identified through the present specification can be provided.

### BRIEF DESCRIPTION OF DRAWINGS

FIG. 1 illustrates a comprehensive cosmetic provision platform system according to an embodiment of the present invention.

FIG. 2 illustrates main components of a comprehensive cosmetic provision platform server.

FIG. 3 illustrates a comprehensive cosmetic provision platform service provided by a comprehensive cosmetic provision platform server according to a contract between a manufacturer and a distributor according to an embodiment of the present invention.

FIG. 4 illustrates a drawing related to determination of cosmetics for a user based on cosmetic use information of other users residing in a smooth distribution area by a distributor.

FIG. 5 is a drawing of illustrating determination of a fee to be charged to a manufacturer and a distributor according to FIG. 3.

FIG. 6 illustrates the hardware configuration of the comprehensive cosmetic provision platform server 100 of FIG. 1.

### BEST MODE

Since the present invention may be applied with various modifications and may have various embodiments, exemplary embodiments and drawings of the present invention are intended to be explained and exemplified. However, these exemplary embodiments and drawings are not intended to limit the embodiments of the present invention to particular modes of practice, and all changes, equivalents, and substitutes that do not depart from the spirit and technical scope of the present invention should be understood as being encompassed in the present invention. Like reference numerals refer to like elements in describing each drawing.

The terms such as “first,” “second,” “A” and “B” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the teachings of the present invention. The term “and/or” includes any or all combinations of one or more of the associated listed items.

It should be understood that when an element is referred to as being “connected to” or “coupled to” another element, the element may be directly connected or coupled to the other element or intervening elements may be present. In contrast, when an element is referred to as being “directly connected to” or “directly coupled to” another element, there are no intervening elements present.

The terms used in the present specification are used to explain a specific exemplary embodiment and not to limit the present inventive concept. Thus, the expression of singularity in the present specification includes the expression of plurality unless clearly specified otherwise in context. Also, terms such as “include” or “comprise” should be construed as denoting that a certain characteristic, number, step, operation, constituent element, component or a combination thereof exists and not as excluding the existence of or a possibility of an addition of one or more other characteristics, numbers, steps, operations, constituent elements, components or combinations thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, the present invention will be described in detail by explaining exemplary embodiments of the invention with reference to the attached drawings.

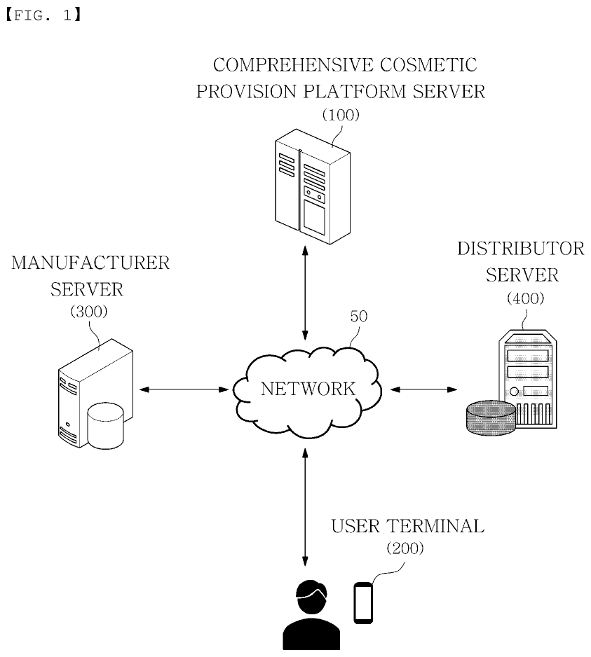

FIG. 1 illustrates a comprehensive cosmetic provision platform system 10 according to an embodiment of the present invention. Referring to FIG. 1, the comprehensive cosmetic provision platform system 10 may include a comprehensive cosmetic provision platform server 100, a user terminal 200, a manufacturer server 300, a distributor server 400, and the like. Operations described below may be performed through a platform (e.g., a web page and/or a kind of application) controlled by the comprehensive cosmetic provision platform server 100.

The comprehensive cosmetic provision platform server 100 may provide a cosmetic recommendation service for a user and a sales promotion service for manufacturers and/or distributors.

The comprehensive cosmetic provision platform server 100 may determine a manufacturer to be brokered through the comprehensive cosmetic provision platform and may proceed and conclude a contract with the determined manufacturer. The comprehensive cosmetic provision platform server 100 may store information about cosmetics of the determined manufacturer in a cosmetic DB.

The comprehensive cosmetic provision platform server 100 may receive manufacturer information and information about cosmetics manufactured by the manufacturer from each of a plurality of manufacturer servers. The comprehensive cosmetic provision platform server 100 may store the received plural information in a manufacturer DB. The comprehensive cosmetic provision platform server 100 may store the received information about cosmetics manufactured by the manufacturer in a cosmetic DB. In other words, the comprehensive cosmetic provision platform server 100 may obtain information about a plurality of cosmetics provided by manufacturers and may store the obtained cosmetic information in the cosmetic DB.

The comprehensive cosmetic provision platform server 100 may receive distributor information from each of a plurality of distributor servers. The comprehensive cosmetic provision platform server 100 may store the received plural distributor information in a distributor DB. The distributor information may include information about the locations of distributor's business sites, the sizes of distributors, distribution stores, and the like. Information about distribution stores may include the locations of distributor's stores, product arrangement styles of the distributor's stores, the number of products that can be placed in the distributor's stores, the types of products distributed or sold in the distributor's stores, and the like.

The comprehensive cosmetic provision platform server 100 may obtain a user's input signal indicating the type of cosmetics desired by a user through a kiosk interlocked with the comprehensive cosmetic provision platform server 100. For example, the comprehensive cosmetic provision platform server 100 may obtain a user's input signal instructing the type of cosmetics including foundation, BB cream, cushion, serum, toner, essence, moisture cream, moisture gel cream, nourishing cream, night cream, and sunscreen through the kiosk interlocked with the comprehensive cosmetic provision platform server 100.

The comprehensive cosmetic provision platform server 100 may obtain a user's face image, and may determine a user's skin condition based on the obtained face image. The comprehensive cosmetic provision platform server 100 may determine cosmetics for the user according to a user's skin condition from among cosmetics, desired by the user, stored in the cosmetics DB, and may provide information about the determined cosmetics to the user through the kiosk and/or the user terminal 200.

The manufacturer server 300 may conclude a cosmetic supply contract with the distributor server 400. The cosmetic supply contract may include information about cosmetic supply prices, cosmetic types, and cosmetic quantity provided to a distributor by a manufacturer.

The distributor server 400 may order cosmetics from the manufacturer server 300 and may manage an inventory of the ordered cosmetics. The distributor server 400 may sell the cosmetics of the manufacturer through distribution stores. The distributor server 400 may settle the sales volume and sales amount of the cosmetics of the manufacturer sold through the distribution stores. The distributor server 400 may provide the settled information about the sales volume and sales amount of the cosmetics of the manufacturer to the comprehensive cosmetic provision platform server 100.

The user terminal 200 may be a communicationable desktop computer, a laptop computer, a notebook, a smart phone, a tablet PC, a mobile phone, a smart watch, a smart glass, an e-book reader, a portable multimedia player (PMP), a portable game console, a navigation device, a digital camera, a digital multimedia broadcasting (DMB) player, a digital audio recorder, a digital audio player, a digital video recorder, a digital video player, and a personal digital assistant (PDA), or the like.

Each of the comprehensive cosmetic provision platform server 100, the user terminal 200, the manufacturer server 300 and the distributor server 400 may be connected to a communication network 50 to transmit and receive data between each other through the communication network 50. For example, various types of wired or wireless networks such as Local Area Network (LAN), Metropolitan Area Network (MAN), Global System for Mobile Network (GSM), Enhanced Data GSM Environment (EDGE), High Speed Downlink Packet Access (HSDPA), Wideband Code Division Multiple Access (W-CDMA), Code Division Multiple Access (CDMA), Time Division Multiple Access (TDMA), Bluetooth, Zigbee, Wi-Fi, Voice over Internet Protocol (VoIP), LTE Advanced, IEEE802.16m, wireless MAN-Advanced, HSPA+, 3GPP Long Term Evolution (LTE), mobile WiMAX (IEEE 802.16e), formerly EV-DO Rev. C (UMB), flash-OFDM, iBurst and MBWA (IEEE 802.20) systems, HIPERMAN, Beam-Division Multiple Access (BDMA), World Interoperability for Microwave Access (Wi-MAX) and 5G may be used as the communication network 50.

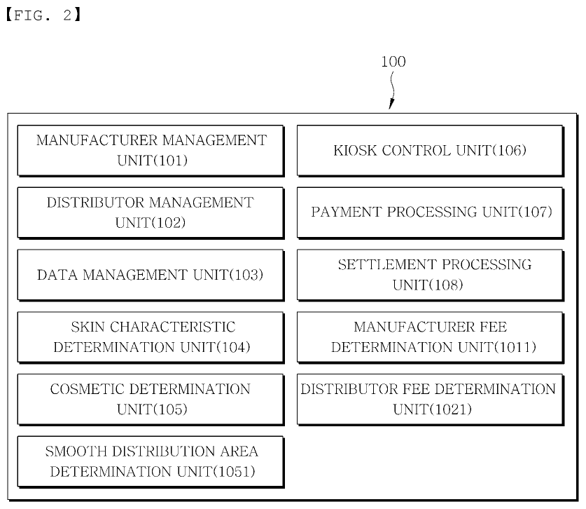

FIG. 2 illustrates main components of the comprehensive cosmetic provision platform server 100.

The comprehensive cosmetic provision platform server 100 may include a manufacturer management unit 101, a distributor management unit 102, a data management unit 103, a skin characteristic determination unit 104, a cosmetic determination unit 105, a kiosk control unit 106, a payment processing unit 107, a settlement processing unit 108, a manufacturer fee determination unit 1011, a distributor fee determination unit 1021, and the like.

The manufacturer management unit 101 may conclude a contract with a manufacturer server, and may obtain manufacturer information; and information about cosmetics provided by the manufacturer from the manufacturer server. The distributor management unit 102 may conclude a contract with the distributor server and may obtain distributor information from the distributor server.

The data management unit 103 may obtain the user's face image from a kiosk installed in a distribution store and may store the obtained face image in a user DB.

The skin characteristic determination unit 104 may determine the skin characteristics of the user at the time of purchase based on the obtained user's face image. The skin characteristics may include a skin type and a skin condition. For example, the skin characteristics may include skin color, the moisture, oil, sebum, pH level, sensitivity, elasticity, and wrinkles of the skin, skin tone, pore conditions, pigmentation, dark circles, redness, keratin conditions, and the like. The skin characteristic determination unit 104 may determine user's skin characteristics through a separate measurement device (e.g., a mask for measurement) in addition to the kiosk and the user terminal 200.

The skin characteristic determination unit 104 may divide the obtained user's face image into a plurality of face regions and may determine the skin characteristics of each of the divided face regions. The plural face regions may include an eye area, a forehead, a cheek, a mouth area, a chin, a neck, and the like.

The skin characteristic determination unit 104 may determine a face region requiring an additional facial image from among the plural face regions, may display the determined face region through the kiosk and/or the user terminal 200, and may obtain an additional facial image of the determined face region through the kiosk and/or the user terminal 200. The skin characteristic determination unit 104 may determine user's skin characteristics based on the previously obtained user's face image and the additional facial image.

The cosmetic determination unit 105 may determine an ingredient and effect necessary for the user based on the user's skin characteristics, and may determine cosmetics including the determined necessary ingredient and effect. The cosmetic determination unit 105 may determine the cosmetics based on cosmetic use information of other users distinguished from the user as well as the determined necessary ingredient and effect. The cosmetic use information of the other users may include purchase reviews, a use period, a use frequency, a return status, and the like. The cosmetic determination unit 105 may generate a recommended list of cosmetics for the user based on the determined cosmetics.

The kiosk control unit 106 may transmit the generated recommendation list and information about cosmetics included in the recommendation list to the kiosk and display the recommendation list and the information about the cosmetics through the kiosk.

The payment processing unit 107 may obtain a payment request signal for the cosmetics from the kiosk, and may perform payment for the cosmetics according to the obtained payment request signal. The settlement processing unit 108 may settle a sales revenue for the cosmetics and may charge a fee to the manufacturer server and the distributor server based on the sales price.

The manufacturer fee determination unit 1011 may determine a preset manufacturer fee as a manufacturer fee to be charged to the manufacturer according to a contract between the comprehensive cosmetic provision platform server 100 and the manufacturer. The distributor fee determination unit 1021 may determine a preset distributor fee as a distributor fee to be charged to the distributor according to a contract between the comprehensive cosmetic provision platform server 100 and the distributor.

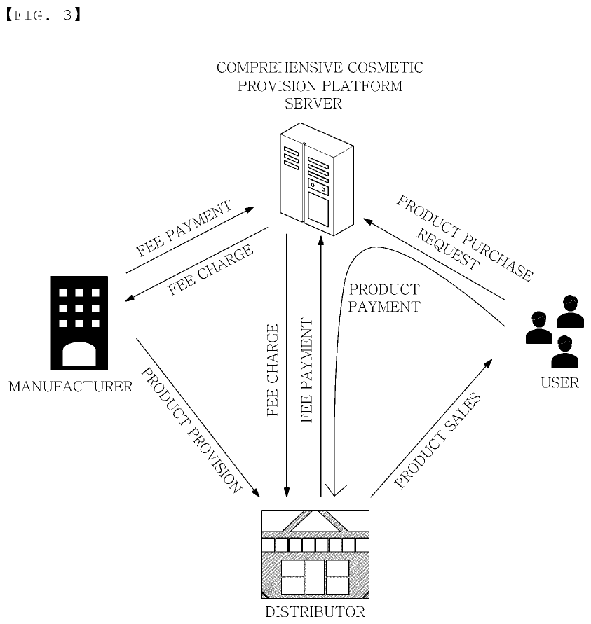

FIG. 3 illustrates a comprehensive cosmetic provision platform service provided by a comprehensive cosmetic provision platform server according to a contract between a manufacturer and a distributor according to an embodiment of the present invention.

The comprehensive cosmetic provision platform server 100 may provide a comprehensive cosmetic provision platform service through a platform use contract with a manufacturer (or manufacturer server 300). The comprehensive cosmetic provision platform server 100 may provide the cosmetic recommendation service for a user and the sales promotion service for a manufacturer as described above, and the manufacturer server 300 may provide a fee for the services to the comprehensive cosmetic provision platform server 100.

The comprehensive cosmetic provision platform server 100 may obtain a user's face image through a terminal (e.g., kiosk) interlocked with the server. The kiosk may include at least one camera for photographing a user's face.

The comprehensive cosmetic provision platform server 100 may receive a cosmetic purchase request and payment request from a terminal (e.g., kiosk) interlocked with the server and/or a user terminal and, accordingly, may process payment. For this, the comprehensive cosmetic provision platform server 100 may include a payment system or may be connected to a separate external payment system or financial system.

As shown in FIG. 3, the comprehensive cosmetic provision platform server 100 may request product delivery to the distributor server 400 according to a user's purchase request or payment request. For this, the comprehensive cosmetic provision platform server 100 may provide user's order details and payment information to the distributor server 400 that provides cosmetics purchased by a user. The distributor server 400 may directly process product delivery and manage a product inventory. A distributor may receive cosmetics from a manufacturer in advance. The comprehensive cosmetic provision platform server 100 may receive product inventory information from the distributor server 400 to store in the data management unit 101 (e.g., product DB).

In another embodiment, the manufacturer server 300 may directly process the inventory management and delivery of manufactured cosmetics without going through the distributor server 400. The comprehensive cosmetic provision platform server 100 may request product delivery to the manufacturer server 300 according to a user's purchase request. For this, the manufacturer server 300 may include a logistics system or may be connected to a separate external logistics system or management system.

The distributor server 400 may process payment for product provision with respect to the manufacturer server 300. The distributor server 400 may process payment for each predetermined cycle with respect to the manufacturer server 300. For example, the distributor server 400 may process payment for products, provided from the manufacturer server 300 for the past month, once a month.

FIG. 3 illustrates the relationship among one manufacturer, one distributor and the comprehensive cosmetic provision platform server 100, but the present invention is not limited thereto. The comprehensive cosmetic provision platform server 100 may be connected to a different number of manufacturers and a different number of distributors.

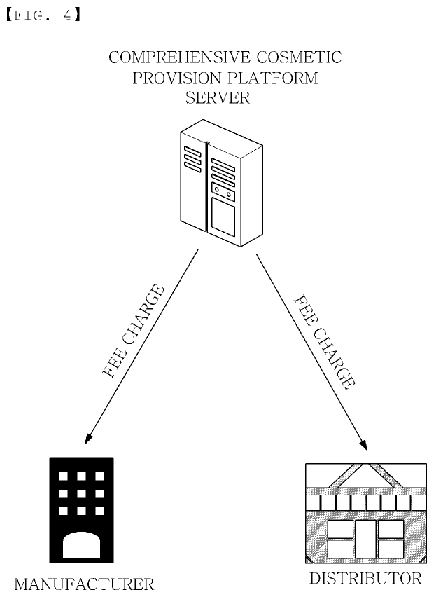

FIG. 4 illustrates a drawing related to determination of cosmetics for a user based on cosmetic use information of other users residing in a smooth distribution area by a distributor.

The cosmetic determination unit 105 may determine the cosmetics based on cosmetic use information of other users residing in a smooth distribution area by a distributor and/or climate information about the smooth distribution area while considering not only skin characteristics determined through a face image, but also cosmetic use information about other users distinguished from the user. In other words, the cosmetic determination unit 105 may determine cosmetics for a user in consideration of the skin characteristics of the user, an environment similar to that of the user and other users belonging to the similar environment. The cosmetic determination unit 105 may determine the cosmetics based on climate information, such as a monthly average temperature, a monthly maximum temperature, a monthly minimum temperature, and a monthly average humidity, on the smooth distribution area obtained from the Meteorological Administration server.

A smooth distribution area determination unit 1051 may determine a smoothly distributable area by the distributor within a range that can be distributed by the distributor. The smooth distribution area determination unit 1051 may calculate a distribution access degree of the distributor to determine the distributable area.

The “distribution access degree” is not a straight-line distance, but may be actually a concept representing a time where a product is distributed through courier delivery from a distributor's business site (e.g., distributor's warehouse, distributor's store) to a delivery destination. The distribution access degree may include distribution time from the time user's payment for a product is completed to the collection processing, trunk loading, trunk unloading, delivery, and delivery completion of the product.

The smooth distribution area determination unit 1051 may determine a distribution access degree (Sₐ) through Equation 1 below.

$${S}_{a} = \{ d + \frac{{\sum }_{k = 1}^{j} ( {a}_{k} \times x + {c}_{k} \times y + {l}_{k} \times z )}{j} \} \times N \qquad [ Equation 1 ]$$

where Sₐ denotes a distribution access degree, Sd denotes a time taken from collection processing of cosmetics to loading on a trunk line, a denotes the length of an arterial road among distribution routes of the k-th cosmetics, b denotes the length of a collection road among distribution routes of the k-th cosmetics, and c denotes the length of a local road among distribution routes of the k-th cosmetics. j denotes the number of deliveries of cosmetics. N is an access factor and may vary depending on the ordered time and day of the week for cosmetics.

x may denote a weight for an arterial road, y may denote a weight for an aggregation road, and z may denote a weight for a local road. x, y, and z are values greater than 0 and less than 1, and since local roads have a longer circulation time per unit distance, y may be greater than x and z may be greater than y.

Here, an arterial road, which is a main road that is the basis of a road network, may refer to a road for connecting important districts between cities or within cities, a collection road, which is a road having a function of handling traffic gathered from other roads, may refer to a road connecting traffic of a local road with an arterial road, and a local road, which is the lowest road in the functional system of the road, may refer to a road through which direct passage occurs in a residential area and/or a commercial area.

In an embodiment, since some users apply for same-day delivery service during courier delivery, the smooth distribution area determination unit 1051 may determine the distribution access degree based on the rate of same-day delivery. The distribution access degree may be shortened as the rate of same-day delivery increases.

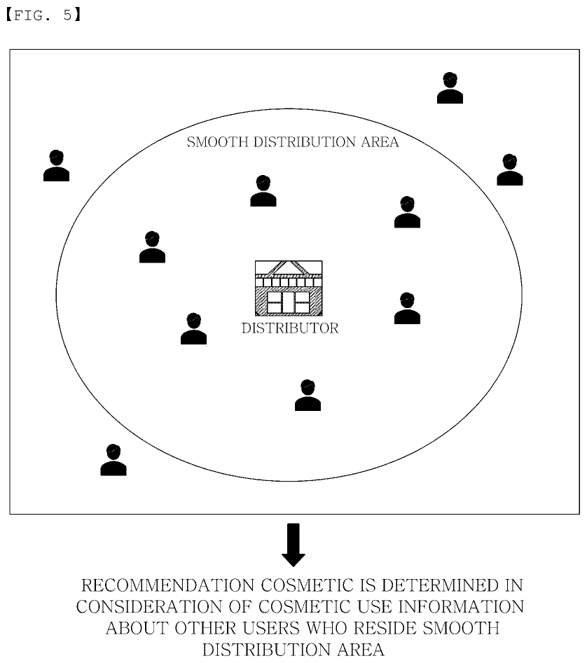

FIG. 5 is a drawing of illustrating determination of a fee to be charged to a manufacturer and a distributor according to FIG. 3.

The manufacturer fee determination unit 1011 may determine a manufacturer fee previously determined according to a contract between the comprehensive cosmetic provision platform server 100 and the manufacturer server 300 as a manufacturer fee to be charged to a manufacturer. For example, the manufacturer fee determination unit 1011 may settle 10% of the sales of the manufacturer at the end of the month and may determine an amount corresponding to the 10% as the manufacturer's fee, according to the contract.

The manufacturer fee determination unit 1011 may determine a manufacturer fee to be charged to the manufacturer based on the sales price, monthly average sales volume, average monthly total sales, monthly average sale ranking, monthly average sales total ranking and/or margin ratio ranking of the cosmetics sold to users through the kiosk.

The manufacturer fee determination unit 1011 may determine a manufacturer's fee to be lower than a preset manufacturer's fee as a cosmetic of a manufacturer sold according to the service provided by the comprehensive cosmetic provision platform server 100 has a higher sale ranking among cosmetics of manufacturers. The manufacturer fee determination unit 1011 may determine a manufacturer's fee to be is higher than a previously determined manufacturer's fee as a cosmetic of a manufacturer sold according to the service provided by the comprehensive cosmetic provision platform server 100 has a lower sale ranking among cosmetics of manufacturers. In other words, since the sale of a cosmetic with a poor sale ranking is promoted according to the service provided by the comprehensive cosmetic provision platform server 100 as a cosmetic of a manufacturer sold according to the service provided by the comprehensive cosmetic provision platform server 100 has a low sale ranking among cosmetics of manufacturers, the manufacturer fee determination unit 1011 may determine a manufacturer's fee to be higher than a preset manufacturer's fee.

The manufacturer fee determination unit 1011 may determine a manufacturer's fee to be higher than a preset manufacturer's fee as a cosmetic of a manufacturer sold according to the service provided by the comprehensive cosmetic provision platform server 100 has a higher margin ratio among cosmetics of manufacturers. The manufacturer fee determination unit 1011 may determine a manufacturer's fee to be lower than a preset manufacturer's fee as a cosmetic of a manufacturer sold according to the service provided by the comprehensive cosmetic provision platform server 100 has a lower margin ratio among cosmetics of manufacturers. In other words, since the manufacturer fee determination unit 1011 induces a cosmetic with a higher margin ratio to be sold by the service provided by the comprehensive cosmetic provision platform server 100, the manufacturer fee determination unit 1011 may determine a manufacturer's fee to be higher than a preset manufacturer's fee.

The distributor fee determination unit 1021 may determine a distributor fee preset according to a contract between the comprehensive cosmetic provision platform server 100 and the distributor server 400 as a distributor fee to be charged to a distributor. For example, the distributor fee determination unit 1021 may settle 10% of the sales of the distributor at the end of the month according to the contract and may determine an amount corresponding to the settled 10% as a distributor fee.

The distributor fee determination unit 1021 may determine a distributor fee to be charged to a distributor based on the utilization of a kiosk installed in a distribution store. The distributor fee determination unit 1021 may determine the kiosk utilization of a distributor based on gross sales growth/decrease rate of a distribution store calculated based on the time that the kiosk was installed in a distribution store, the number of face images obtained through the kiosk, the adoption rate of cosmetics recommended and displayed through the kiosk, an installation position of the kiosk in a distribution store, and the like. The adoption rate of the cosmetics may mean a ratio of cosmetics displayed through the kiosk leading to purchase.

The distributor fee determination unit 1021 may determine that the kiosk utilization is low as the location where the kiosk is installed is farther from a payment desk of a distribution store. In other words, since the probability that customers using a distribution store will approach a payment desk to pay is high, and the probability that customers will approach a location far from the payment desk is low, it may be determined that the utilization of the kiosk is low as the installation location of the kiosk is farther from the payment desk.

The distributor fee determination unit 1021 may determine a distributor fee to be lower than a preset distributor fee as the kiosk utilization of the distributor is high. Conversely, the distributor fee determination unit 1021 may determine a distributor fee to be higher than a preset fee.

The distributor fee determination unit 1021 may determine a distributor fee based on the types of other products that are distinguished from the cosmetic and are being sold at the distribution store.

The distributor fee determination unit 1021 may determine a distributor fee based on a product relevance degree between a cosmetic and other products that are being sold in the distribution store. For example, the distributor fee determination unit 1021 may determine a fee to be high when other products that are being sold in a distribution store have a low relevance degree with the cosmetic, and a fee may be determined to be low when other products that are being sold in a distribution store have a high relevance degree with the cosmetic. In other words, since there is a high probability that a cosmetic will be sold according to the service provided by the server 100 if products highly related to a cosmetic are sold through a distributor according to the so-called furniture street effect that you have to go to the furniture street to buy furniture, a distributor fee may be determined to be lower than a preset distributor fee so as to increase the benefits to the distributor. Conversely, since the probability that a cosmetic will be sold according to the service provided by the comprehensive cosmetic provision platform server 100 is low when a distributor sells products having a low relevance degree with a cosmetic, a distributor fee may be determined to be higher than a preset fee so as to minimize the benefits to the distributor.

The distributor fee determination unit 1021 may determine a product relevance degree between each of the other products between a cosmetic based on whether the other products are food and beverage products, fluid components-containing products, skin care-related products, medical products, beauty-related products, health-related products, etc. The distributor fee determination unit 1021 may calculate a first score according to whether the other products are food and beverage products, a second score according to whether the other products are fluid components-containing products, a third score according to whether the other products are skin care-related products, a fourth score according to whether the other products are medical products, a fifth score according to whether the other products are beauty-related products, and a sixth score according to whether the other products are health-related products. For example, the distributor fee determination unit 1021 may calculate a preset score (e.g., 1 point) if it corresponds to each of the factors and may calculate a preset score (e.g., 0 point) if it corresponds to each of the factors and, based on the calculated scores, may determine the product relevance degree.

The distributor fee determination unit 1021 may determine a distributor fee to be lower than a preset distributor fee when the calculated product relevance degree is a threshold relevance degree or more. The distributor fee determination unit 1021 may determine a distributor fee to be higher than a preset distributor fee when the calculated product relevance degree is less than a threshold relevance degree.

The distributor fee determination unit 1021 may determine a distributor fee to be charged to the distributor server 400 based on a product relevance degree between a cosmetic and other products sold by a distributor, the kiosk utilization, and the like.

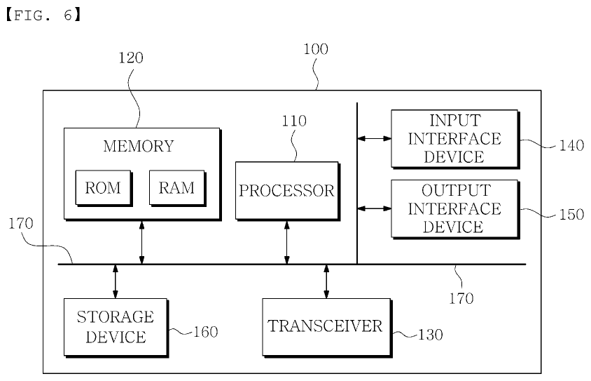

FIG. 6 illustrates the hardware configuration of the comprehensive cosmetic provision platform server 100 of FIG. 1.

Referring to FIG. 6, the comprehensive cosmetic provision platform server 100 may include at least one processor 110; and a memory for storing instructions that instruct the at least one processor 110 to perform at least one operation.

The at least one operation may include at least some of the above-described operations or functions of the comprehensive cosmetic provision platform server 100 and may be implemented in the form of instructions to be performed by the processor 110.

Here, the at least one processor 110 may mean a central processing unit (CPU), a graphics processing unit (GPU), or a dedicated processor where the methods according to embodiments of the present invention are performed. Each of the memory 120 and the storage device 160 may be configured of at least one of a volatile storage medium and a non-volatile storage medium. For example, the memory 120 may be one of a read only memory (ROM) and a random access memory (RAM), and the storage device 160 may be a flash-memory, a hard disk drive (HDD), a solid state drive (SSD), various memory cards (e.g., micro SD card), or the like.

In addition, the comprehensive cosmetic provision platform server 100 may include a transceiver 130 for performing communication through a wireless network. In addition, the comprehensive cosmetic provision platform server 100 may further include an input interface device 140, an output interface device 150, a storage device 160, and the like. Each of the components included in the comprehensive cosmetic provision platform server 100 may be connected by a bus 170 to communicate with each other. FIG. 6 illustrates the comprehensive cosmetic provision platform server 100 as an embodiment, but the present invention is not limited thereto. For example, a plurality of user terminals may include the components of FIG. 6.

The methods according to the embodiments of the present disclosure may be implemented in the form of a program command that can be executed through various computer means and recorded in a computer-readable medium. The computer-readable medium can store program commands, data files, data structures or combinations thereof. The program commands recorded in the medium may be specially designed and configured for the present disclosure or be known to those skilled in the field of computer software.

Examples of a computer-readable recording medium may include hardware devices such as ROMs, RAMs and flash memories, which are specially configured to store and execute program commands. Examples of the program commands may include machine language code created by a compiler and high-level language code executable by a computer using an interpreter and the like. The hardware devices described above may be configured to operate as at least one software module to perform the operations of the invention, and vice versa.

In addition, the above-described method or apparatus may be implemented by combining all or part of constructions or functions thereof, or the constructions or functions may be separately implemented.

Although the present invention has been described above with reference to the embodiments of the present invention, those skilled in the art may variously modify and change the present invention without departing from the spirit and scope of the present invention as set forth in the claims below.

### DESCRIPTION OF SYMBOLS

100: comprehensive cosmetic provision platform server
200: user terminal
300: manufacturer server
400: distributor server

1. A comprehensive cosmetic provision platform server for providing a service that recommends a cosmetic to a user, the comprehensive cosmetic provision platform server comprising:
a memory; and
a processor, wherein the processor is configure to:
obtain information about a manufacturer and information about the cosmetic provided by the manufacturer from a manufacturer server,
obtain distributor information including information about the locations of distributor's business sites, the sizes of distributors, and distribution stores from a distributor server,
obtain a face image of the user taken through a camera included in a kiosk in a distribution store of the distributor server,
determine an ingredient and effect necessary for the user based on the user's skin characteristics and determine a cosmetic comprising the ingredient and effect necessary for the user based on the determined skin characteristics including wrinkles of the skin, pore conditions, pigmentation, dark circles, redness, and troubles,
determine the cosmetic based on cosmetic use information of other users who are distinguished from the user, wherein the cosmetic use information of the other users includes purchase reviews, a use period, a use frequency, and a return status,
determine a smooth distribution area, which is a smoothly distributable area, within a range that can be distributed by the distributor,
determine the cosmetic based on climate information comprising a monthly average temperature, a monthly maximum temperature, a monthly minimum temperature, and a monthly average humidity, on the smooth distribution area that can be distributed by the distributor,
generate a recommendation list of cosmetics for the user based on the determined cosmetic,
transmit the generated recommendation list and information about cosmetics included in the recommendation list to the kiosk and display the recommendation list and the information about the determined cosmetic through the kiosk,
obtain a payment request signal for the cosmetic from either the kiosk or a payment terminal of the distributor and perform payment for the cosmetic according to the obtained payment request signal, and
calculate a sales amount for the cosmetic and a fee to the manufacturer server and the distributor server based on the sales amount,
wherein the smooth distribution area is determined according to an equation below,
$${S}_{a} = \{ d + \frac{{\sum }_{k = 1}^{j} ( {a}_{k} \times x + {c}_{k} \times y + {l}_{k} \times z )}{j} \} \times N$$
wherein Sₐ denotes the distribution access degree, d denotes a time taken from collection processing of cosmetics to loading on a trunk line, aₖ denotes the length of an arterial road among distribution routes of the k-th cosmetics, cₖ denotes the length of a collection road among distribution routes of the k-th cosmetics, and lₖ denotes the length of a local road among distribution routes of the k-th cosmetics, j denotes the number of deliveries of cosmetics, N is an access factor and may vary depending on the ordered time and day of the week for cosmetics, x denotes a weight for an arterial road, y denotes a weight for an aggregation road, and z denotes a weight for a local road, x, y, and z are values greater than 0 and less than 1, and y is greater than x and z is greater than y, and
wherein the arterial road, which is a main road that is the basis of a road network, refers to a road for connecting important districts between cities or within cities, the collection road, which is a road having a function of handling traffic gathered from other roads, refers to a road connecting traffic of a local road with an arterial road, and the local road, which is the lowest road in the functional system of the road, refers to a road through which direct passage occurs in a residential area or a commercial area.

2. The comprehensive cosmetic provision platform server according to claim 1, wherein the processor is configured to:
calculate a first sales amount paid for the cosmetic at the kiosk according to the payment request signal obtained from the kiosk; and a second sales amount paid for the cosmetic at the payment terminal according to the payment request signal obtained from the payment terminal of the distributor, and
the second sales amount is a sales amount in which payment for the cosmetic is performed by the payment terminal within a predetermined time after information about the cosmetic is displayed through the kiosk.

3. (canceled)

4. The comprehensive cosmetic provision platform server according to claim 1, wherein the processor is configured to:
determine the manufacturer fee for the manufacturer server based on a monthly average sales ranking, selling price, and margin rate of the cosmetic payment-requested by the user.

5. The comprehensive cosmetic provision platform server according to claim 2, wherein the processor is configured to:
determine a distributor fee for the distributor server based on the calculated first and second sales amounts, a product relevance degree between the cosmetic and other products that are being sold in the distribution store and distinguished from the cosmetic, and utilization of the kiosk.
